(19) United States
(12) Patent Application Publication
(10) Pub. No.: US 2008/0096986 A1
(43) Pub. Date: Apr. 24, 2008

(54) Method for synthesizing a catalyst based on anisotropic metallic nanoparticles by a micellar means

(76) Inventors: Cecile Thomazeau, Brignais (FR); Denis Uzio, Belleville (FR); Catherine Verdon, Marly Le Roi (FR); Bogdan Harbuzaru, Lyon (FR); Gilles Berhault, Lyon (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/598,194

(22) Filed: Nov. 13, 2006

(30) Foreign Application Priority Data
Nov. 14, 2005 (FR) 05/11.531

(51) Int. Cl. B01J 23/70; B01J 23/40; C07C 7/144; C07C 5/32; C07C 27/06; C01B 31/20; C07C 7/163; B22F 1/00

(52) U.S. Cl. 518/716; 502/337; 502/338; 502/339; 502/325; 75/343; 585/275; 585/276; 585/899; 585/430; 585/434; 423/437.2; 518/719; 518/715; 428/546

(57) ABSTRACT

The invention relates to a process for preparation of anisotropic metallic nanoparticles comprising at least: a) One stage that is brought into contact with an aqueous solution that comprises at least one source of a metal that is selected from the columns 8, 9 or 10 of the periodic table and at least one soluble surfactant, b) One stage for formation of anisotropic metallic nanoparticles of at least one of said metals, by adding at least one reducing agent to the solution that is obtained in stage a), c) Said particles are: c1) separated from liquid and optionally dried, or c2) deposited on a substrate by impregnation with the suspension obtained in stage b) or after resuspension of the nanoparticles that are obtained at the end of stage c1), whereby the substrate is an oxide of unordered texture, and after separation of the possible residual liquid, the material that is obtained is dried at a temperature that is less than or equal to 120° C., and said solid is subjected to a reduction treatment by hydrogen at a temperature that is less than or equal to 100° C.

### FIELD OF THE INVENTION

This invention relates to a process for preparation of anisotropic metallic nanoparticles, in particular deposited on a substrate. Said particles can be used as a catalyst.

### PRIOR ART

The metals, in particular the transition metals, have been used for many years for their ability to activate a certain number of molecules such as hydrocarbons, hydrogen, oxygen, or else carbon monoxide. The catalytic properties of the metals with regard to these molecules have as their object a large number of works that made it possible to demonstrate their versatility based on metal, conditions of implementation but also their physico-chemical characteristics.

The phenomena for activation by chemisorption, i.e., the phenomena that are governed by the adsorption of reagents and the chemical interactions between said reagents and the metallic particle, depend on the physico-chemical properties of the exposed metallic surfaces. The size of the metallic particle and the coordinance of the atoms on the surface of this particle are two characteristics to be taken into consideration to obtain catalysts that have optimum chemisorptive properties, i.e., catalysts that are suitable for adsorbing the reagents and for interacting chemically with said reagents.

As far as the size of the metallic particle is concerned, the latter is generally adjusted based on the application in question.

It is known that the metallic nanoparticles are able to adopt different stable shapes based on the number of atoms that constitute them or chemical interactions with their environment, i.e., for example, with the reaction atmosphere. The metallic particles that are used in heterogeneous catalysis exhibit morphologies having a marked isotropic nature, adopting stable geometric shapes that depend on the number of atoms that constitute the particle. Among these geometric shapes, it is possible to cite the icoshedral shape for small particles that are generally smaller in size than about 10 Angstrom, and the truncated cubooctahedral shape for larger particles.

One object of the invention is to provide a method for synthesizing a catalyst that is based on metallic nanoparticles, which may or may not be deposited on a substrate, making it possible to control the shape of these nanoparticles and in particular making it possible to prepare nanoparticles, which may or may not be in substrate form, exhibiting an anisotropic morphology, i.e., exhibiting a mean shape factor of less than 1.

A certain number of methods for synthesizing anisotropic metallic nanoparticles are already well known to one skilled in the art. By way of example, it is possible to cite the lithographic processes and the vapor phase growth techniques.

The methods for synthesizing anisotropic nanoparticles implementing matrices are also known by one skilled in the art. In these methods, the material is generated in situ inside the matrix that imparts to it a morphology by replicating that of the matrix. The nanometric channels of the matrix are filled with a solution that is destabilized by a sol-gel process, or else by an electrochemical process. A reduction is then necessary to form the nanoparticles inside the matrix. The inadequate yield of anisotropic nanoparticles and the necessity to eliminate the matrix without impairing said nanoparticles are drawbacks of these replication techniques.

Methods for synthesizing nanoparticles in solution in an organic medium, for example in alcohol or in polyol, in which the growth of the nanoparticles can be controlled by selective agent adsorption in a single stage are also known to one skilled in the art. A reduction is generally carried out by reflux, i.e., at the boiling point of the organic medium, and the organic phase is used both as solvent for the selective adsorption agent, generally a polymer, and as a reducing agent for the metallic precursor. These methods are described in “Y. Sun, Y. Xia, Advanced Materials, 2002, 14, 833-837.” They exhibit the drawback of using slightly reducing organic compounds that should be activated at high temperature. The yields that are obtained are relatively low. In addition, the operating parameters that make possible a monitoring of the nanoparticle shapes are not very numerous. It is a matter primarily of the temperature in a relatively narrow range of the ratio between the concentrations of selective adsorption agent and metallic precursor.

Among the methods for synthesis in solution in an organic medium, some of them make possible a monitoring of the growth of the metallic nanoparticles by selecting a precursor of the metal being considered that has ligands that promote the reduction and the growth in a given direction. In these methods of synthesis, a decomposition of these organometallic precursors is implemented as is described in “Chaudret et al., C. R. Chimie, 6, 2003, 1019-1034.” By way of example, it is possible to cite the use of thiophenol ligands or hexadecylamine for the synthesis of nanotubes and nanothreads made of platinum respectively, as is described in “Gomez et al., Chem. Comm. 2001, 1474.”

It is known to synthesize isotropic nanoparticles in an aqueous or organic medium by a micellar means. These methods benefit from the organization of surfactant molecules in the form of micelles, whereby the latter play the role both of template and nanoreactor. Surfactant molecules are defined as any molecule that contains a hydrophilic polar portion and a hydrophobic hydrocarbon chain. The micelles are formed at the interface between two environments, one aqueous and the other organic. The normal micelles, formed in the presence of an organic minority phase and an aqueous majority phase, are generally distinguished from reverse micelles, the latter being formed in the presence of a majority organic solution and a minority aqueous phase. The reverse micelles appear as good structuring agents of nanoparticles. Their size increases linearly with the amount of water added to the system, as is described in “M. P. Pileni et al., Chemical Physics Letters, 1985, 118, 414.” This adjustment of the size of micelles by adding water makes possible the monitoring of the final size of the generally spherical nanoparticles. These methods for preparation by a micellar means of isotropic metallic nanoparticles gave rise to multiple studies that are known to one skilled in the art, and a good monitoring of the size of the isotropic nanoparticles can now be obtained, as is described in “Jana et al., Journal of the American Chemical Society, 2003, 125, 14280-14281.”

By implementation of these methods for synthesis by a micellar means in an organic environment, it is known to synthesize nanoparticles that exhibit an anisotropic nature and that have a mean shape factor, F, less than or equal to 0.70, in particular nanoparticles of cylindrical shape. Actually, it is known that cylindrical micelles can be used as an agent for structuring cylindrical copper nanoparticles as is described in “M. P. Pileni, Langmuir, 17, 2001, 7476-7486.” However, this micellar approach that uses reverse micelles is considered for copper, silver or gold, metals that by nature exhibit a strong suitability for reduction.

The European Patent EP 1 338 361 describes a method for producing nanoparticles by a micellar means, by producing reverse micelles on metal salts. In this method, only reverse micelles are used, which involves use of a large amount of an organic solvent.

### SUMMARY OF THE INVENTION

A process for preparation of anisotropic nanoparticles of metals of columns 8 to 10 of the periodic table is now proposed, said process making it possible to obtain a good monitoring of the morphology.

The invention also relates more specifically to a process for preparation of anisotropic metallic nanoparticles comprising at least:
a) One stage for bringing into contact an aqueous solution comprising at least one source of a metal that is selected from the columns 8, 9 or 10 of the periodic table and at least one surfactant,
b) One stage for formation of anisotropic metallic nanoparticles by adding at least one reducing agent to the solution that is obtained in stage a),
c) Said particles are:
   c1) Separated from the liquid, and optionally dried
   or c2) Deposited on a substrate by impregnation with the suspension that is obtained in stage b) or after resuspension of the nanoparticles obtained at the end of stage c1), whereby the substrate is an oxide of unordered texture, and after separation of the possible residual liquid, the material that is obtained is dried at a temperature that is less than or equal to 120° C.,
and said solid is subjected to a treatment of reduction by hydrogen at a temperature that is less than or equal to 100° C.

The synthesis method that is found makes it possible to produce anisotropic nanoparticles that have a mean shape factor, F, that is less than 1, in particular nanoparticles that have a marked anisotropic nature, i.e., having a mean shape factor of less than 0.7, preferably less than 0.5, and even less than 0.25.

Said shape factor being defined by the formula F=(4*II*S)/P², S and P being measured by transmission electron microscopy, S being the surface area of the particle measured in a characterization plane, P being the perimeter of the particle measured in this same plane, and in which said substrate has an unordered porosity. The shape factor can be calculated from electron-microscope measurements according to the methods that are described in “Coster, M., Chermant, J. L., Précis d'analyse d'images [Synopsis of Image Analysis], Eds CNRS, 1985.” The mean shape factor, F, is determined by statistical analysis by observing the statistical counting rules known to one skilled in the art.

The nanoparticles may have sizes from several angstroms to several hundred nanometers. Advantageously, the average size (i.e., the size according to the largest dimension of the particles) of the nanoparticles is between 2 and 500 nanometers, or between 5 and 200 nm, preferably more than 10 nm, and, for example, from 10 to 500 nm, or else 10 to 200 nm, or 10 to 120 nm. It was possible to note, surprisingly enough, that sizes of more than 10 nm unexpectedly led to good catalytic performance levels.

The invention therefore relates to containing the anisotropic metallic nanoparticles that are obtained by the process according to the invention and also to a suspension that contains them. It also relates to the nanoparticles that are obtained by the process according to the invention, in solid form and separated from the liquid. It also relates to a substrate that comprises anisotropic metallic nanoparticles that are obtained by the process according to the invention.

The suspension above, the nanoparticles, in solid form and separated from the liquid, and the substrate that comprises anisotropic metallic nanoparticles, as defined above, can be used as catalysts.

The invention also relates to the applications of the solids that are described above for the separation or the adsorption of molecules, the storage of gas.

The catalyst can be used for the catalytic transformation of organic molecules; in particular, it is used for the catalytic reforming of hydrocarbons, the total or selective hydrogenation, the dehydrocyclization, the dehydrogenation of hydrocarbons, the Fischer-Tropsch synthesis, or the total or selective oxidation of the carbon monoxide.

### BRIEF DESCRIPTION OF THE FIGURES

FIG. 1 shows, in a nonlimiting manner, a photograph by electronic microscopy of anisotropic nanoparticles of platinum obtained according to Example 1.

FIG. 2 shows, in a nonlimiting manner, a photograph by electron microscopy of anisotropic nanoparticles of palladium obtained according to Example 2.

### DETAILED DESCRIPTION OF THE INVENTION

The invention relates to a process for preparation of anisotropic metallic nanoparticles comprising at least:
a) One stage that is brought into contact with an aqueous solution that comprises at least one source of a metal that is selected from the columns 8, 9 or 10 of the periodic table and at least one soluble surfactant,
b) One stage for formation of anisotropic metallic nanoparticles of at least one of said metals, by adding at least one reducing agent to the solution that is obtained in stage a),
c) Said particles are:
   c1) separated from liquid and optionally dried,
   or c2) deposited on a substrate by impregnation with the suspension obtained in stage b) or after resuspension of the nanoparticles that are obtained at the end of stage c1), whereby the substrate is an oxide of unordered texture, and after separation of the possible residual liquid, the material that is obtained is dried at a temperature that is less than or equal to 120° C.,
and said solid is subjected to a reduction treatment by hydrogen at a temperature that is less than or equal to 100° C.

Thus, during stage a), normal micelles are formed. The normal micelles are also described as direct micelles. The normal or direct micelles are distinguished from reverse micelles in that they are formed in the presence of an organic minority phase (surfactant) and an aqueous majority phase (by volume relative to the organic phase).

The normal micellar path, relative to the reverse micellar path, makes it possible to work in majority aqueous phase, which is advantageous from an environmental point of view. The normal micellar path also makes possible, relative to the reverse micellar path, the synthesis of a larger amount of catalyst for the same volume of solution and obtaining a larger yield of anisotropic particles.

The detection of micelles is done, for example, by the measurement of surface tension and more generally by any technique that is known to one skilled in the art.

Metals and Sources of Metals (Columns 8, 9 and 10)

The metal of the nanoparticles that are produced is selected from among the metals that belong to columns 8, 9 and 10 of the periodic table. The metal of the nanoparticles that are produced is preferably selected from among nickel, cobalt, iron, ruthenium, platinum, palladium, and iridium, and preferably cobalt, nickel, platinum and palladium. In contrast to gold, silver or copper, which by nature are greatly suited to reduction, it was found that the metals listed above could, surprisingly enough, be used in the synthesis method of the invention.

The method of the invention also makes it possible to produce a catalyst based on nanoparticles of several metals. Thus, nanoparticles of a metal selected in the columns 8 to 10 are defined as nanoparticles of at least one metal that is selected from said columns.

The anisotropic metallic nanoparticle can comprise a single metallic element or several metallic elements. In the case of nanoparticles that comprise several metallic elements, the latter can be combined in any way known to one skilled in the art. It may involve any mixture, such as an alloy, a solid solution or any structure that comprises a core and/or a shell.

The metal source can be any salt of a precursor of the metal being considered that has a degree of oxidation of the metal that is greater than 0. This salt may be a simple halide or oxide or hydroxide of the metal being considered or else a salt that combines a halide with an alkaline compound, an alkaline-earth compound, an amine group or an ammonia group. This salt can also be a nitrate, a nitrite or a sulfate of the metal that is considered by itself or in combination with an amine group. This salt can also comprise at least one organic ligand.

For example, it will be possible to use the following as a palladium source: palladium chloride, palladium bromide, palladium iodide, potassium hexachloropalladate, ammonium hexachloropalladate, potassium tetrabromopalladate, potassium tetrachloropalladate, ammonium tetrachloropalladate, sodium hexachloropalladate, sodium tetrachloropalladate, palladium nitrate, palladium nitrite, diaminepalladium nitrite, palladium sulfate, tetraaminepalladium nitrate, palladium dichlorodiamine, palladium acetate, and palladium acetylacetonate.

The metal source content in the solution that is subjected to a reduction stage can be encompassed between 10⁻⁵ and 1 mol/liter, preferably between 5 10⁻⁵ and 10⁻¹ mol/liter, and more preferably between 10⁻⁴ and 10⁻² mol/liter. It is calculated based on the content to be obtained in the final product.

The Surfactant

The solution that is formed, during stage a), comprises a surfactant.

It is possible to use one or more surfactants, able to be used simultaneously or successively. The content of surfactant in the solution that is obtained during stage a) is generally between 0.2 and 2 mol/liter, preferably between 0.5 and 2 mol/liter, and advantageously between 0.7 and 1.5 mol/liter.

With the CTAB surfactant, the work is most often done between 0.8 and 1.2 mol/liter. The concentrations that range from 0.7 to 1.5 mol/liter (and more particularly between 0.8 and 1.2 mol/liter with the CTAB) promote the formation of anisotropic micelles.

The surfactant concentration is selected so that the aqueous phase remains in the majority (by volume relative to the organic phase) so as to form normal micelles.

The surfactant is brought into solution while being stirred at a temperature such that it remains soluble, i.e., between the ambient temperature and 100° C., and preferably generally between the ambient temperature and 60° C., the temperature depending on the nature of the surfactant. For example, with the tetraoctylammonium bromide, it is around 30° C.

The reaction time for stage a) generally goes from 1 minute to 48 hours according to the metal/reducing agent pair.

Surfactant is defined as any organic compound that has a hydrophilic polar group and a hydrophobic hydrocarbon chain.

Advantageously, the surfactant can be any compound of general formula RxR′yXzYd in which:
x and y are between 0 and 4,
z and d are between 0 and 2,
R is hydrogen or a hydrocarbon chain that comprises between 1 and 25 carbon atoms, preferably between 8 and 18,
R′ is hydrogen or any aliphatic, aromatic or heteroaromatic group that is known to one skilled in the art,
X is carbon or nitrogen or a cationic element that is taken from the alkaline group, the alkaline-earth group, or an ammonia group, and
Y is an anionic element such as a halide, nitrate, nitrite or sulfate.

It is possible to use, in addition, a co-surfactant. The co-surfactant that is used can be any organic compound that can induce the formation of cylindrical micelles, such as: n-hexanol, n-heptanol, or tetraoctyl ammonium bromide.

The introduction of the surfactant can be carried out by any means known to one skilled in the art.

A preferred process for preparation comprises:
The separate dissolutions of surfactant, on the one hand, and of the metal source, on the other hand,
Followed by the mixing of at least one portion of dissolved surfactant and at least one portion of the dissolved metal source.

It makes possible a better control of the dissolution of the surfactant, which induces a greater degree of self-organization in the vicinity of the micelles.

The surfactant can advantageously be used in ranges of concentrations for which cylindrical micelles are formed, whereby these ranges are generally known to one skilled in the art. For example, it is possible to use as surfactant:
Hexadecyltrimethylammonium bromide with a content of between 0.2 and 1.5 mol, or
Sodium dodecyl sulfate with a content of between 0.9 and 2 mol.

The preceding concentration ranges advantageously make possible the formation of cylindrical micelles.

The Reducing Agent

A reducing agent is added to the solution that is formed in stage a); this stage b) for addition of the reducing agent can be implemented consecutively or at the same time as stage a).

The reducing agent that is used may be of inorganic nature or organic nature. It is not hydrogen. The reducing agent is preferably selected from the group that consists of:
Hydrazine, hydroxylamines, alkaline hydrides and borohydrides, for inorganic reducing agents, and
Carboxylic acids, alcohols, polyols, aldehydes, ketones, and their ions for the organic reducing agents.

These chemical reducing agents have the advantage, relative to the hydrogen, of facilitating the implementation, of carrying out a reduction more homogeneously and of obtaining a better monitoring of the reduction speed.

The addition of the reducing agent during stage b) is advantageously carried out while being stirred directly or gradually. The amount of reducing agent added can vary from 0.1 to 10 equivalents per metal equivalent, preferably 1 to 5.

Stage b) is carried out at a temperature for which the surfactant remains soluble, generally between the ambient temperature and 100° C., preferably between the ambient temperature and 60° C., whereby this temperature depends on the nature of the surfactant.

Preferably, if the reducing agent has a protonated group (carboxylic acid, alcohol, . . . ), during stage b), a base, which is more preferably soda, is also added. The addition of the base makes it possible to obtain the corresponding anion by deprotonation, the latter exhibiting a stronger reducing capacity. The base (for example, soda) is preferably added in an equimolar quantity relative to the reducing agent.

Preferably, during stage b), an inorganic salt is added. This makes it possible to monitor the interaction between the growth agent and the anisotropic nanoparticle in formation.

This salt can be of any type of inorganic compound that comprises halide anions such as chloride, bromide, fluoride, nitrates, nitrites, or sulfates. Preferably, the inorganic salt that is added in stage b) is selected from the group that consists of the alkali halides and the alkaline-earth compounds.

The amount of added inorganic salt is generally between 10⁻⁴ and 0.5, preferably between 5 10⁻⁴ and 0.1, and more preferably between 10⁻³ and 0.05 mol/liter of the total volume of the solution that is obtained in stage b).

Obtaining Materials

In one method, according to stage b) for formation of anisotropic metallic nanoparticles, the latter can be separated from the liquid by any means known to one skilled in the art. By way of example, these means can include centrifuging, for example at 5000 rpm for 45 minutes. They are optionally dried at a temperature that is less than or equal to 120° C., preferably less than or equal to 80° C., or else at 50° C. under an inert atmosphere or in air. A solid that consists essentially of metallic particles thus is obtained.

According to one method, the anisotropic metallic nanoparticles that are obtained during stage b) are deposited on a substrate by impregnation and advantageously by dry impregnation.

Thus, the depositing stage may take place starting from the solution that is obtained in stage b) or by resuspension of the metallic nanoparticles that are obtained in stage b) (in an amount corresponding to the contents desired for the material or the suspension to be used) and a contact of said solution with a substrate.

After impregnation, the substrate is separated from the possible residual liquid and dried.

The substrate preferably is an oxide of unordered texture. Substrate that has an unordered texture is generally defined as a substrate that has no particular structural property, no texture with a particular geometry, no mesostructurings (i.e., the substrates that have a texture ordered at the scale of the pores).

The substrate is generally based on at least one refractory oxide that is selected from among alumina, silica, silica-alumina, magnesium oxide, zirconium oxide, thorium oxide, or titanium oxide, by itself or mixed with one another. Preferably, the substrate is alumina, silica or silica-alumina.

The substrate can also be a carbon, a silico-aluminate, a clay or any other compound that is known for being used as a substrate, as defined in the invention.

Generally, the substrate has a BET surface area of between 5 and 300 m²/g.

The substrate can be used in powder form or can be worked into the shape of balls, extrudates, trilobes, powder or monoliths.

On this substrate, the anisotropic nanoparticles can be isolated with respect to one another or can form networks, such as, for example, intergrowths of threads, fractal structures or metallic foams.

In a general way, it is possible to introduce at the substrate at least one element that is selected from the group that is formed by:
The elements of column 13 of the periodic table,
The elements of column 14 of the periodic table,
The alkaline metals, preferably lithium, sodium or potassium, and/or the alkaline-earths, preferably magnesium, calcium, strontium or barium, and/or
The halogens,
The elements of column 11.

These elements are generally introduced after the nanoparticles are deposited on the substrate and after possible drying, possible reduction treatment (as will be described later).

The optional addition of at least one alkaline metal and/or at least one alkaline-earth can be carried out so as to obtain a content of alkaline metals and/or alkaline-earths in the catalyst that is between 0 and 20% by weight, preferably between 0 and 10% by weight, and more preferably between 0.01 and 5% by weight.

The optional addition of at least one halogen can be carried out so as to obtain a halogen content in the catalyst of between 0 and 0.2% by weight.

According to the invention, the solid or the suspension is subjected to a treatment of reduction by the hydrogen at a temperature of less than 600° C., preferably less than 400° C., or equal to 100° C., preferably less than 80° C., or, better, 50° C. When other elements are added to the substrate material, this treatment may take place before said additions or after.

The Products that are Obtained

The invention therefore makes it possible to produce solid materials that contain anisotropic metallic nanoparticles, as well as suspensions that contain said particles.

In these materials or suspension, generally at least 50% by weight, preferably at least 70% by weight, even more preferably at least 90% by weight of metals of groups 8 to 10 is in the form of anisotropic metallic nanoparticles.

By way of example, the nanoparticles that are obtained by the method of the invention can have a morphology such as rods, threads, and even a tubular morphology, solid or hollow tubes. The metallic nanoparticles of the catalyst that is obtained by the method of the invention can also have a cylindrical morphology that it is possible to define by a length-to-width ratio. In this case, the length-to-width ratio can be equal to 5 or 10, which corresponds to a shape factor that is respectively equal to about 0.43 and 0.25.

In the case of a material (in particular used as a catalyst), and in particular based on substrate nanoparticles, the metal content is preferably between 0.01 and 50% by weight, preferably between 0.01 and 35% by weight. Generally, and in particular for the use as catalyst, the process according to the invention can be applied for the contents of metals that are usually used.

Use of the Products that are Obtained

The anisotropic metallic nanoparticles and the products that contain them (such as the suspensions or solid materials described above), products obtained by the process according to the invention, can advantageously be used as catalysts.

The solid materials that comprise a substrate and anisotropic metallic nanoparticles that are obtained by the process according to the invention can also be used as absorbents or in separation membranes. Thus, the invention also relates to a process for the separation or the adsorption of molecules, the storage of gas with said materials.

They can also be used as absorbents or in separation membranes. Thus, the invention also relates to a process for the separation or the adsorption of molecules, the storage of gas with said materials.

More preferably, the catalyst (preferably in substrate form), obtained by the method of the invention, is used for the catalytic transformation of organic molecules. Such uses are generally encountered in the field of refining and petrochemistry.

These are, for example, the catalytic reforming of hydrocarbons, total or selective hydrogenation, dehydrocyclization, dehydrogenation of hydrocarbons, Fischer-Tropsch synthesis, or the total or selective oxidation of carbon monoxide.

The catalysts can be used, for all the processes cited for catalytic transformation of organic molecules, under the operating conditions and with the feedstocks to be treated that are those of the standard processes. Several indications will be provided below.

The uses in separation can implement dense metallic membranes that use the intrinsic properties of certain metals, such as the dissolution and the diffusion of molecules, such as hydrogen or oxygen, in their metallic network and thus make possible the purification of gas flow, as is described in the Japanese Patent Application JP2002153740.

Thus, for example, a membrane purification unit will be operated at a temperature of between 300° C. and 600° C., and at a pressure of between 0.6 and 2 MPa, relative, to separate a gaseous effluent that is the hydrogen of very high purity that is generally more than 95% and preferably more than 99%.

The uses that are linked to the storage of gas, such as hydrogen, can also make use of metallic systems that use the adsorption capacities of multimetallic formulations, as described in the International Patent Application WO2004027102. The storage of hydrogen can be carried out at an H2 pressure of between 0 and 10 atm, at a temperature of between 0 and 100° C.

The catalytic reforming of the hydrocarbons is generally intended to produce a fuel with a high octane number by using platinum-based catalysts, as is described in the U.S. Pat. No. 5,562,817 or U.S. Pat. No. 6,315,892). The catalyst can also be used for the production of aromatic hydrocarbons.

The temperature is generally between 400° C. and 600° C., preferably 480° C. to 580° C., and the pressure is between 0.1 and 3.5 MPa, preferably 0.2 and 1.8 MPa, for a volumetric flow rate of 0.1 and 10 volumes of liquid feedstock per volume of catalyst, with an H2/hydrocarbon ratio of between 1 and 20, preferably 1 to 6.

The selective hydrogenation is generally intended to purify the effluents of a steam-cracking device or of catalytic cracking by using palladium-based catalysts as is described in “W. K. Lam, L. Lloyd, Oil & Gas Journal, pp. 66-70, March 1972.” It will be possible to refer to the conditions of use of the Patent EP0899012 for the selective hydrogenation of diolefins or acetylenic compounds. The conditions that are generally used for this type of transformation are a mean temperature of between 25 and 200° C., a pressure of between 0.1 and 10 MPa and a hydrogen to hydrocarbon molar ratio of between 1 and 150.

For total hydrogenation (EP0899012) of hydrocarbons comprising alkyne, diene or olefin groups, or aromatic compounds, the operation is performed at a mean temperature of between 10 and 400° C. and under a pressure of between 0.1 and 10 MPa.

The Fisher-Tropsch synthesis is intended to produce higher hydrocarbons from a synthesis gas that comprises carbon monoxide and hydrogen.

It will be possible to refer to U.S. Pat. No. 6,214,890. Generally, the conversion of the syngas takes place under 0.1-15 MPa, 150-350° C., 100-20,000 volumes of gas/volume of catalyst and per hour, with a feedstock that has an H2/CO ratio=1.2 to 2.5, whereby the process can be implemented in a fixed bed or in slurry.

The object of the reactions for oxidation of the carbon monoxide is to purify the gaseous effluents that contain carbon monoxide, optionally in the presence of other compounds such as hydrogen. For example, reference will be made to FR2867464.

The total oxidation reaction of the CO can be carried out at a high temperature of between 350° C. and 550° C. The total pressure of the conversion unit of the CO at high temperature will be between 0.6 and 2 MPa, relative.

The reaction of the oxidation of the CO can be carried out at a low temperature of between 180° C. and 260° C. The total pressure of the section for conversion of the CO at low temperature will be between 0.6 and 2 MPa, relative.

The selective oxidation reaction of the CO is carried out for total pressures of between atmospheric pressure and 50 bars, preferably between atmospheric pressure and 20 bars, and even more preferably between atmospheric pressure and 10 bars. The temperature is between 20 and 200° C., preferably between 50 and 150° C., and even more preferably between 70 and 130° C. The CO/O2 molar ratio is between 0.5 and 5, preferably between 1 and 4. The CO:H2 ratio by weight is between 0.5:50 and 5:50, preferably between 1:50 and 3:50.

The dehydrocyclization (EP 1252259) generally takes place under pressures of 0.1 to 4 MPa (preferably 0.3-0.8 MPa for the regenerative reforming and for the process for production of aromatic compounds, 0.02-2 MPa for the dehydrogenation of the C3-C22 paraffins), 300-800° C. (preferably 400-700° C. and preferably 480-600° C. for the regenerative reforming and for the process for production of aromatic compounds; 400-550° C. for the dehydrogenation of the C3-C22 paraffins), volumetric flow rates from 0.1 to 10 h−1 and preferably 1-4 h−1 and with variable H2/hydrocarbon ratios (recycled hydrogen/hydrocarbons (mol) of 0.1 to 10 and preferably 3-10, and more particularly 3-4 in the first reactor used for the regenerative reforming and 4-6 for the process for production of aromatic compounds; H2/hydrocarbons of 10-100 liters of hydrocarbons per liter of catalyst and per hour for the dehydrogenation of C3-C22 paraffins).

### EXAMPLES

The process for preparation of nanoparticles according to the invention is illustrated in the following examples. The performance levels of the catalysts that comprise nanoparticles that are obtained directly by the method of the invention are compared to those of the catalysts that are obtained by methods of the prior art. These examples have an illustrative nature that does not limit the scope of the invention.

The morphology of the nanoparticles of the catalysts that are used within the framework of these examples was the object of a characterization by transmission electron microscopy. The electron microscope that was used was the Jeol 2010© model marketed by the JEOL Company. This microscope model has an acceleration tension of 200 kV, a spatial resolution of 0.2 nm, and a detection limit of metallic substrate particles on the order of 0.7 nm.

The shape factor F is determined by using image processing software IMAGIST© developed by “Image Processing and Analysis, Princeton Gamma Tech.”

Before carrying out the characterization by electron microscopy, the catalyst samples were prepared by following a procedure including dissolving in ethanol, the depositing of a drop of solution on the analysis grid, the drying and the introduction of said grid into the microscope.

### Example 1

### Synthesis of Anisotropic Platinum Nanoparticles on an Alumina Substrate

### Catalyst A, in Accordance with the Invention

A solution of metallic precursor was prepared by dissolution of 0.72 g of [Pt(NH₃)₄]Cl₂ in 100 ml of water thermostated to 35° C. The cetyl trimethylammonium bromide (CTAB) (43.7 g) was added to the metallic precursor solution to obtain a concentration of 1.2 mol/L. The reducing agent (NaBH₄, 76 mg in 5 ml of H₂O) was added while being stirred vigorously. The solution was stirred by remaining at 35° C. for 30 minutes, a period at the end of which the solution turns black. After reduction, the solution was diluted in water. Platinum nanothreads were thus obtained.

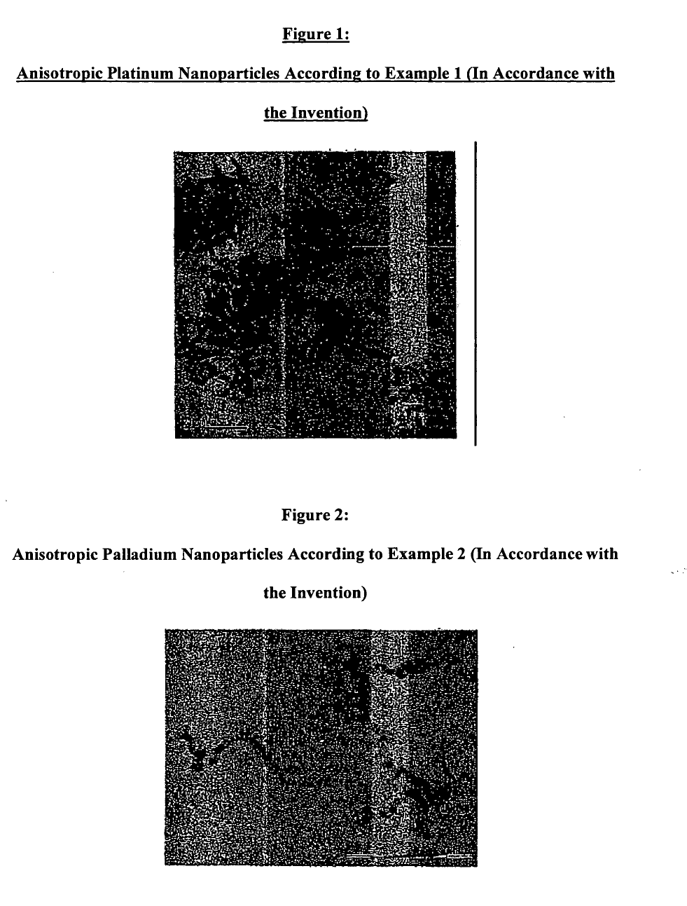

A photograph by electron microscopy of these nanoparticles is provided in FIG. 1. The mean dimensions of these nanothreads are about 20 nm in length and 3 nm in width. The mean shape factor of the nanoparticles is equal to 0.4. These nanothreads were washed several times with hot water and separated by centrifuging.

After redispersion in water, the platinum nanoparticles were deposited on alumina (Al₂O₃) by dry impregnation. The catalyst was then dried for one night at 60° C. The thus prepared catalyst A contains 0.3% by weight of platinum.

### Example 2

### Synthesis of Anisotropic Palladium Nanoparticles on an Alumina Substrate

### Catalyst B, in Accordance with the Invention

A metallic precursor solution was prepared by the dissolution of 0.49 g of [Pd(NH₃)₄]Cl₂ in 100 ml of water thermostated to 35° C. The cetyl trimethylammonium bromide (43.7 g) was added to the solution to obtain a concentration of 1.2 mol/L. The reducing agent (NaBH₄, 76 mg in 5 ml of H₂O) was added while being stirred vigorously. The solution was stirred for 5 minutes, a period at the end of which it turns black. After reduction, the solution was diluted in water.

A photograph by electron microscopy of these nanoparticles is provided in FIG. 2. The palladium nanothreads were then washed with warm water and separated by centrifuging. The nanothreads have a particular “pearl necklace” shape with dimensions of 80-100 nm in length and 10 nm in thickness. The mean shape factor of the nanoparticles is equal to 0.25.

After redispersion in water, the Pd nanoparticles are deposited on Al₂O₃ by dry impregnation. The catalyst was then dried for one night at 60° C. The thus prepared catalyst B contains 0.3% by weight of palladium.

### Example 3

### Synthesis of Anisotropic Cobalt Nanoparticles on an Alumina Substrate

### Catalyst C, in Accordance with the Invention

A metallic precursor solution was prepared by dissolution of 0.72 g of Co(NO₃)₂ in 100 ml of water thermostated to 35° C. The cetyl trimethylammonium bromide (CTAB) (43.7 g) was added to the metallic precursor solution to obtain a concentration of 1.2 mol/l. The reducing agent (NaBH₄, 76 mg in 5 ml of H₂O) was added while being stirred vigorously. The solution was stirred for 30 minutes, a period at the end of which the solution turns black. After reduction, the solution was diluted in water.

Co nanothreads were thus obtained. Their mean dimensions are about 25 nm in length and 2 nm in width. The mean shape factor of the nanoparticles is equal to 0.2. These nanothreads were then washed several times with warm water and separated by centrifuging.

After redispersion in water, the cobalt nanoparticles were deposited on alumina (Al₂O₃) by dry impregnation. The catalyst was then dried for one night at 60° C. The thus prepared catalyst C contains 5% by weight of cobalt.

### Example 4

### Synthesis of Isotropic Palladium Nanoparticles on an Alumina Substrate

### Catalyst D, not in Accordance with the Invention

A platinum catalyst on an alumina substrate and containing isotropic palladium particles was prepared by impregnation with an excess of toluene solution that contains the precursor Pd(acac)₂. The catalyst was then dried for one night at 120° C. and calcined in air at 350° C. for 2 hours.

The thus prepared catalyst D contains 0.3% by weight of palladium. The size of the substrate particles is between 2 and 5 nm, and their morphology, observed by transmission electron microscope, is of the spherical type, with a shape factor F that is equal to 1.

### Example 5

### Synthesis of Isotropic Platinum Nanoparticles on an Alumina Substrate

### Catalyst E, not in Accordance with the Invention

A platinum catalyst on an alumina substrate containing isotropic platinum particles was prepared by impregnation with an excess of toluene solution containing the precursor Pt(acac)₂. The catalyst was then dried for one night at 120° C. and calcined in air at 350° C. for 2 hours.

The thus prepared catalyst 3 contains 0.3% by weight of platinum. The size of the substrate particles is between 2 and 5 nm; their morphology, observed by transmission electron microscopy, is of the spherical type, with a shape factor F equal to 1.

### Example 6

### Synthesis of Isotropic Cobalt Nanoparticles on an Alumina Substrate

### Catalyst F, not in Accordance with the Invention

A cobalt-based catalyst on an alumina substrate comprising isotropic cobalt particles was prepared by dry impregnation of a Co(NO₃)₂ solution, drying for one night at 120° C. and calcination at 200° C.

The thus prepared catalyst F comprises 5% by weight of cobalt. The size of the particles is between 3 and 9 nm with a spherical-type morphology (shape factor F equal to 1).

### Example 7

### Catalytic Test for Hydrogenation of 1,3-Butadiene

The hydrogenation of the 1,3-butadiene was carried out in liquid phase (n-heptane) in a perfectly-stirred, “Grignard”-type batch reactor under a constant pressure of 20 bars of hydrogen and a thermostated temperature of 20° C. The products of the reaction were analyzed by gas phase chromatography. The catalytic activities expressed in mol of H₂/min/g of metal, determined by tracking the pressure drop, are indicated in Table 1 below. The selectivities for 1-butene were measured at 80% conversion of 1,3-butadiene. Before the test, the catalysts A and B were treated in advance under hydrogen at 50° C. The catalyst D is treated in advance under H2 at 200° C. The catalyst E is treated in advance under H2 at 550° C.

TABLE 1
Activities and Measured Selectivities
for Hydrogenation of 1,3-Butadiene

| | Activity | Selectivity |
|---|---|---|
| | Mol/min/g of Metal | 1-Butene (%) |
| Catalyst A (Pt) | 1.5 | 63 |
| Invention | | |
| Catalyst E (Pt) | 0.6 | 60 |
| For Comparison | | |
| Catalyst B (Pd) | 3.5 | 60 |
| Invention | | |
| Catalyst D (Pd) | 2.5 | 60 |
| For Comparison | | |

Catalyst A, whose metallic particles have a mean shape factor, F, that is equal to 0.4, has a catalytic hydrogenation activity of 1,3-butadiene (expressed per gram of platinum) and a selectivity of 1-butene that are higher than those of catalyst E, which has isotropic metallic particles with a shape factor F that is equal to 1.

The catalyst B, whose metallic particles have a mean shape factor F that is equal to 0.25, has a catalytic hydrogenation activity of 1,3-butadiene (expressed per gram of palladium) that is higher than that of the catalyst D, which has isotropic metallic particles with a shape factor F that is equal to 1.

### Example 8

### Catalytic Test for Conversion of Synthesis Gas

The reaction for conversion of synthesis gas was implemented in a continuous fixed-bed reactor under a constant pressure of 2 MPa of hydrogen and a temperature of 220° C. The hourly volumetric flow rate was from about 1500 h⁻¹, and the H₂/CO molar ratio was kept at 2.

The catalytic activities expressed in terms of the conversion of carbon monoxide are indicated in Table 2 below. Prior to the catalytic test, the catalyst C was treated under a stream of hydrogen at 100° C.; the catalyst F was treated under a stream of hydrogen at 400° C.

TABLE 2
Activities and Measured Selectivities for Conversion
of Synthesis Gas into Hydrocarbons

| | CO | | |
|---|---|---|---|
| | Conversion | Yield of | % of |
| | % | Hydrocarbons > C₅ | CH₄ |
| Catalyst C (Co) | 32 | 66 | 12 |
| Invention | | | |
| Catalyst F (Co) | 32 | 54 | 14 |
| For Comparison | | | |

For the same conversion of carbon monoxide, the catalyst C, whose metallic particles have a mean shape factor F that is equal to 0.2, has a higher selectivity of heavy hydrocarbons than the one observed for the catalyst F, whose metallic particles are isotropic with a shape factor F that is equal to 1.

### Example 9

### Catalytic Test for Transformation of a Petroleum Fraction

### Catalytic Reforming

The catalytic reforming reaction was carried out, in the presence of hydrogen (H₂/feedstock molar ratio=5), in a fixed-bed continuous reactor on a petroleum fraction whose characteristics are as follows:
Density at 20° C.: 0.75 kg/dm³
Research octane number: 60
Paraffin content: 51%
Naphthene content: 36%
Aromatic compound content: 13%

The experimental conditions were as follows:
A total pressure of 0.3 MPa,
A temperature of 500° C., and
A feedstock flow rate of 3 kg/kg of catalyst.

During the test, the temperature was gradually adjusted to keep the octane number constant. Prior to the catalytic test, the catalyst A is treated under a stream of hydrogen at 100° C.; the catalyst E is treated under a stream of hydrogen at 550° C. The catalytic performance levels that are obtained, after 10 hours of operation, are indicated in Table 3 below.

TABLE 3
Activities and Measured Selectivities for Catalytic Reforming

| | Desired | Yield of | Yield of Aromatic |
|---|---|---|---|
| | Octane | Reformate | Compounds |
| | Number | (% by Weight) | (% by Weight) |
| Catalyst A | 102 | 93 | 74 |
| (Pt) | | | |
| Invention | | | |
| Catalyst E | 102 | 92 | 72 |
| (Pt) For | | | |
| Comparison | | | |

Catalyst A, whose metallic particles have a mean shape factor, F, equal to 0.4, makes it possible to improve the yield of aromatic compounds relative to the catalyst E that has isotropic metallic particles.

### Example 10

### Catalytic Test for Dehydrogenation of Paraffins C₁₀ to C₁₄

The dehydrogenation reaction of paraffins C₁₀ to C₁₄ was carried out in a flushed-bed reactor, at 450° C., under a pressure of 0.29 MPa, with a hydrogen to hydrocarbon molar ratio of 6, and an hourly volumetric flow rate of 20 h⁻¹. An input of 2000 ppm of water was maintained throughout the test.

Catalysts A and E underwent, prior to the test, the following preparation stages:
100 g of catalyst was dissolved in 450 ml of distilled water. 0.64 g of tin dichloride was added to the solution in the presence of hydrochloric acid. The suspension was left for 4 hours while being stirred.
The catalyst was dried, then again dissolved in 100 ml of distilled water, whose pH was adjusted to 1.3 by the addition of nitric acid containing 3.45 g of lithium nitrate. The suspension was left for 3 hours while being stirred.
The catalysts were dried at 150° C. for 2 hours in air
The catalyst A is then treated under hydrogen at 100° C.; the catalyst E is then treated under hydrogen at 550° C.

The catalytic performance levels measured after 100 hours of operation are indicated in Table 4 below.

TABLE 4
Activities and Measured Selectivities
for Dehydrogenation of Paraffins

| | Invention | For Comparison |
|---|---|---|
| | Catalyst A | Catalyst E |
| Conversion of n-Paraffins | 8.27 | 8.0 |
| C10-C14 (%) | | |
| Yield of Olefins | 7.6 | 7.10 |
| C10-C14 (%) | | |
| Yield of Aromatic Compounds | 0.14 | 0.28 |
| C10-C14 (%) | | |
| Yield of Diolefins | 0.24 | 0.25 |
| C10-C14 (%) | | |
| Yield of Iso-Paraffins | 0.22 | 0.22 |
| C10-C14 (%) | | |
| Yield of Other Products | 0.07 | 0.15 |
| C10− and C14+ (%) | | |

In the iso-conversion of the paraffins C₁₀ to C₁₄, the catalyst A, whose metallic particles have a mean shape factor, F, equal to 0.4, makes it possible to obtain a higher olefin yield and a lower aromatic compound yield than those obtained with the catalyst E that has isotropic metallic particles.

### Example 11

### Catalytic Test for Selective Oxidation of Carbon Monoxide

The selective oxidation reaction of the carbon monoxide was carried out in a flushed-bed reactor, at atmospheric pressure, at temperatures of between 70 and 130° C. The reaction mixture CO:H₂:O₂:He comprised 2% by weight of carbon monoxide and 50% by weight of H₂, with a CO/O₂ ratio of 3. The catalytic performance levels are provided in Table 5 below.

TABLE 5
Measured Activities for Selective Oxidation
of Carbon Monoxide at 100° C.

| | Invention | For Comparison |
|---|---|---|
| | Catalyst A (Pt) | Catalyst E (Pt) |
| Conversion of CO (%) | 100 | 100 |
| Selectivity of O₂ (%) | 47 | 40 |

The selectivity measured for the conversion of the oxygen of catalyst A, whose metallic particles have a mean shape factor F that is equal to 0.4, is higher than that measured for the catalyst E, which has isotropic metallic particles.

### Example 12

### Hydrogen Storage Capacity Test

The hydrogen storage capacity test was carried out on a Rubotherm pressurized thermobalance, making it possible to follow the kinetics of absorption of hydrogen in a temperature range of between 20 and 1000° C., and pressure between 1 and 50 bars. The tested catalyst was installed in the measuring nacelle; the reactor was then purged by being evacuated and flushed with hydrogen and then placed under pressurized hydrogen. The heat cycles were then carried out to determine the kinetics of adsorption (hydridation) and the kinetics of desorption (decomposition) of hydrogen.

TABLE 6
Hydrogen Storage Capacities for T = 150° C., PH₂ = 1 Bar

| | Catalyst B | Catalyst D |
|---|---|---|
| | (Pd) | (Pd) For |
| | Invention | Comparison |
| H₂ Storage Capacity | 0.62 | 0.40 |
| (% by Weight) | | |

The hydrogen adsorption capacity measured for the catalyst B, whose metallic particles have a shape factor that is equal to 0.25, is greater than the hydrogen adsorption capacity measured for the catalyst D, whose metallic particles have a shape factor that is equal to 1.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius, and all parts and percentages are by weight, unless otherwise indicated.

The entire disclosures of all applications, patents and publications, cited herein and of corresponding French Application No. 05/11,531, filed Nov. 14, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for preparation of anisotropic metallic nanoparticles comprising at least the following stages:
a) providing an aqueous solution comprising at least one source of at least one metal selected from columns 8, 9 or 10 of the periodic table and at least one soluble surfactant,
b) formation of forming a suspension of anisotropic metallic nanoparticles of said at least one metal, by adding at least one reducing agent to said aqueous solution,
c1) separating said anisotropic metallic nanoparticles from liquid and optionally drying the resultant separated anisotropic metallic nanoparticles,
or c2) depositing on a substrate by impregnation with the suspension that is obtained in stage b) or after resuspension of the nanoparticles that are obtained at the end of stage c1), whereby the substrate is an oxide of unordered texture, and separating residual liquid, from the substrate, and drying the resultant substrate at a temperature that is less than or equal to 120° C.,
and subjecting the resultant nanoparticles from (C1) or the substrate from (C2) to a reduction treatment by hydrogen at a temperature that is less than or equal to 100° C.

2. A process according to claim 1, in which the nanoparticles have a mean size of between 10 and 500 nm.

3. A process according to claim 1, wherein the at least one metal is nickel, cobalt, iron, ruthenium, platinum, palladium, or iridium.

4. A process according to claim 3, in which the at least one metal is cobalt, nickel, palladium or platinum.

5. A process according to claim 1, in which the reducing agent is any of:
hydrazine, hydroxylamines, alkaline hydrides and borohydrides, as inorganic reducing agents, or
the carboxylic acids, alcohols, polyols, aldehydes, ketones and their ions as organic reducing agents.

6. A process according to claim 1, in which a base is added to said aqueous solution during stage b) with the reducing agent.

7. A process according to claim 6, in which 0.9 to 1.1 equivalents of base/reducing agent equivalent is/are added.

8. A process according to claim 1, in which 0.1 to 10 equivalents of reducing agent/metal equivalent is/are added.

9. A process according to claim 1, in which during stage b), an inorganic salt is added in an amount of between 10⁻⁴ and 0.5 mol/liter of the total volume of the solution that is obtained in stage b).

10. A process according to claim 9, in which the inorganic salt that is added to stage b) is an alkali halide or alkaline-earth compounds.

11. A process according to claim 1, wherein the aqueous solution that comprises the metal source and the surfactant in stage (a) is formed by:
separate dissolutions of the surfactant, and of the metal source,
followed by a mixing of at least one portion of the dissolved surfactant and at least one portion of the dissolved metal source.

12. A process according to claim 1, in which during stage b), anisotropic metallic nanoparticles that have a shape factor F of less than 0.7 are formed.

13. A process according to claim 1, in which at least 50% of the metallic particles are in the form of anisotropic metallic nanoparticles.

14. A process according to claim 13, in which at least one element that is selected from the group that is formed by:
The elements of column 13 of the periodic table,
The elements of column 14 of the periodic table,
The alkaline metals, preferably lithium, sodium or potassium, and/or the alkaline-earths, or magnesium, calcium, strontium or barium, and/or
The halogens,
The elements of column 11.
is introduced at the substrate.

15. A catalyst of comprising a substrate and anisotropic metallic nanoparticles obtained by the process according to claim 1.

16. In the catalytic reforming of hydrocarbons, the improvement wherein the catalyst is according to claim 15.

17. In a catalytic total or selective hydrogenation, the improvement wherein the catalyst is according to claim 15.

18. A process according to claim 17, in which stage c) is carried out at a temperature of less than 80° C.

19. In the catalytic dehydrocyclization of hydrocarbons, the improvement wherein the catalyst is according to claim 15.

20. In the catalytic dehydrogenation of hydrocarbons, the improvement wherein the catalyst is according to claim 15.

21. In the Fischer-Tropsch catalytic synthesis, the improvement wherein the catalyst is according to claim 15.

22. In a catalytic total or selective oxidation of carbon monoxide, the improvement wherein the catalyst is according to claim 15.

23. In a process for the storage of gas using comprising contacting the gas with a solid material the improvement wherein the solid comprises the anisotropic metallic nanoparticles obtained according to claim 1.

24. In a process for the separation or the adsorption of molecules, the improvement wherein the process is conducted in contact with anisotropic metallic nanoparticles obtained according to claim 1.

25. Anisotropic metallic nanoparticles obtained by the process of claim 1.
